(19) United States
(12) Patent Application Publication
(10) Pub. No.: US 2022/0096319 A1
(43) Pub. Date: Mar. 31, 2022

(54) MUSCLE MASSAGE DEVICE

(71) Applicant: Hao YI, Guangdong (CN)

(72) Inventor: Hao YI, Guangdong (CN)

(21) Appl. No.: 17/081,962

(22) Filed: Oct. 27, 2020

(30) Foreign Application Priority Data
Sep. 30, 2020 (CN) 202022219275.8

(51) Int. Cl. A61H 23/02

(52) U.S. Cl. CPC A61H 23/0254; A61H 2201/5043; A61H 2201/0153

(57) ABSTRACT

The present application discloses a muscle massage device including a shell, a high-frequency hitting unit disposed on the shell and an electric pulse unit disposed on the shell. Due to the cooperation of the electric pulse unit and the high-frequency hitting unit of the muscle massage device disclosed by the present application, EMS electric pulse massage and high-frequency vibration massage are effectively combined; the shell is disposed to be of a bilateral symmetry structure which is in smooth transition, so that multi-angle free massage is effectively realized; and a flexible buffer part is disposed between a support fixing part and a motor fixing seat, so that power stability is realized, meanwhile, noise is effectively reduced, and the sense of use is improved.

### BACKGROUND OF THE INVENTION

The present application relates to the technical field of muscle training equipment and in particular to a muscle massage device.

With the social development, people pay more and more attention to the improvement of physical quality, and more and more people join a sports or fitness team. After sports or fitness practice, muscle generally needs to be relaxed to improve and stabilize a sports or fitness effect, and thus, a fascial gun emerges. Compared with a traditional foam roller, the fascial gun is capable of more deeply and precisely relaxing muscular fascia.

An existing fascial gun is incapable of favorably meeting various muscle training demands of people due to single function. Meanwhile, the existing fascial gun is of a gun-type structure and includes a handle and a massage head vertical or approximately vertical to a set angle of the handle, and due to restrictions of the gun-type structure, the existing fascial gun is large in size, heavy and inflexible in use angle.

### BRIEF SUMMARY OF THE INVENTION

The purpose of the present application is to at least solve one of the above-mentioned technical problems to a certain extent.

The present application provides a muscle massage device, including:

a shell;

a high-frequency hitting unit, disposed on the shell;

an electric pulse unit, disposed on the shell; and

a control unit, disposed on the shell and connected with the high-frequency hitting unit and the electric pulse unit, wherein the control unit optionally controls the high-frequency hitting unit and the electric pulse unit to work according to a preset mode, and the high-frequency hitting unit and the electric pulse unit are not allowed to work at the same time;

the shell is provided with a first holding part and a second holding part disposed at intervals; the electric pulse unit includes:

a pulse generation mechanism, disposed inside the shell;

a first electrode sheet, disposed on the first holding part; and

a second electrode sheet, disposed on the second holding part, wherein the pulse generation mechanism is connected with the first electrode sheet and the second electrode sheet, and a pulse current is generated and output when the first electrode sheet and the second electrode sheet are in contact with the skin of a human body after being electrified;

the high-frequency hitting unit includes:

a massage head, at least partially disposed outside the shell; and

a reciprocating driving mechanism, disposed inside the shell and connected with the massage head;

the shell is provided with a reciprocating channel on a position corresponding to the reciprocating driving mechanism; and the reciprocating driving mechanism is configured to drive the massage head to do high-frequency reciprocating motion in an axial direction of the reciprocating channel.

Further, the first electrode sheet and the second electrode sheet are respectively disposed at outer sides of the first holding part and the second holding part; and inner sides of the first holding part and the second holding part are provided with corrugated structures suitable for being held by fingers of a human body.

Further, the shell is provided with a first end and a second end corresponding to each other, the first holding part and the second holding part are located at the first end of the shell and are disposed left and right at intervals, the reciprocating channel is located at the second end of the shell, and an outer contour of the shell is of an axisymmetric structure in bilateral symmetry relative to a central axis of the reciprocating channel.

Further, the first end of the shell is provided with a first grip and a second grip, the first grip and the second grip respectively form the first holding part and the second holding part; and an avoiding gap is formed between the first grip and the second grip at the first end of the shell, and a third holding part corresponding to the massage head is formed at the avoiding gap in the first end of the shell.

Further, the reciprocating driving mechanism includes:

a motor, fixedly disposed inside the shell, wherein the motor includes a motor main body and a motor shaft extending out of the motor main body and capable of rotating, and a central axis of the motor shaft is vertical to the central axis of the reciprocating channel;

an eccentric wheel, fixedly connected to the motor shaft, wherein the side, far away from the motor main body, of the eccentric wheel is provided with an eccentric shaft, and a central axis of the eccentric shaft is parallel to and deviated from the central axis of the motor shaft;

a linkage rod, wherein the linkage rod is provided with a first linkage end and a second linkage end, and the first linkage end is rotatably connected to the eccentric shaft; and

a reciprocating part, wherein the reciprocating part is provided with a first driving end and a second driving end, and the first driving end of the reciprocating part is fixedly connected to the second linkage end of the linkage rod; the massage head is fixedly connected to the second driving end of the reciprocating part; and the reciprocating part is at least partially disposed in the reciprocating channel and is capable of doing reciprocating motion in the axial direction of the reciprocating channel under the drive of the motor.

Further, the reciprocating driving mechanism further includes a motor support configured to fix the motor inside the shell; the motor support includes:

a motor accommodating part, wherein the motor accommodating part is provided with a motor accommodating cavity and a motor shaft through hole, an inner contour of the motor accommodating cavity is identical with an outer contour of the motor main body, and the motor shaft through hole is configured to be penetrated by a motor shaft; and

a support fixing part;

the inside of the shell is provided with a motor fixing seat, the motor support is fixedly disposed on the motor fixing seat by the support fixing part, and a flexible buffer part is disposed between the support fixing part and the motor fixing seat.

Further, the support fixing part and the motor fixing seat are matched to be provided with a locking structure, and the locking structure includes:

at least one first locking notch disposed on the support fixing part; and

at least one second locking notch disposed on the motor fixing seat, wherein the position of the first locking notch corresponds to the position of the second locking notch, and the first locking notch and the second locking notch support against each other.

Further, the electric pulse unit further includes a first electric pulse interface and a second electric pulse interface connected with the pulse generation mechanism, the first electric pulse interface and the second electric pulse interface are configured to be connected with an external electrode sheet so as to further provide a pulse current output for the external electrode sheet.

Further, the muscle massage device further includes a power supply unit connected with the control unit, wherein the power supply unit includes:

a rechargeable battery disposed inside the shell;

an electric energy input interface connected with the rechargeable battery;

and

an electric energy output interface connected with the rechargeable battery.

Further, the control unit includes:

a master control unit;

an instruction unit connected with the master control unit; and

a display unit connected with the master control unit;

the master control unit includes:

an electric pulse control module configured to control the electric pulse unit to work, wherein at least one electric pulse working mode is preset for the electric pulse control module, the electric pulse working mode is embodied as an output mode of the pulse current, a plurality of electric pulse working gears are set for each of the electric pulse working modes, and the electric pulse working gears are embodied as the output intensity of the pulse current; and

a high-frequency hitting control module configured to control the high-frequency hitting unit to work, wherein at least one high-frequency hitting working mode is preset for the high-frequency hitting control module, the high-frequency hitting working mode is embodied as a hitting mode of the massage head, a plurality of high-frequency hitting working gears are further set for each of the high-frequency hitting working modes, and the high-frequency hitting working gears are embodied as the hitting frequency of the massage head;

the instruction unit includes:

a mode switching module, configured to control the electric pulse control module or the high-frequency hitting control module to be started and control the electric pulse control module or the high-frequency hitting control module to work according to a preset working mode; and

a gear adjusting module, configured to adjust a working gear of the electric pulse control module or the high-frequency hitting control module in the current working mode;

the display unit includes:

a working mode display module, configured to embody a currently selected working mode; and

a working parameter display module, configured to embody working parameters in the current mode.

The present application has the beneficial effects that due to the cooperation of the electric pulse unit and the high-frequency hitting unit on the shell, EMS electric pulse massage and high-frequency vibration massage are effectively combined, an electric pulse which may be adapted to a human body is generated by the pulse generation mechanism, these current signals may be recognized by the human body as bioelectric signals of the human body to further stimulate muscle contraction, so that superficial muscles and deep muscles may be trained at the same time; meanwhile, the massage head vibrates to impulse muscular fascia, so that a great deal of creatine generated by the human body due to fatigue caused by sports, work and life may be effectively dissipated, blood circulation may be promoted, and muscles may be relaxed, massaged and trained; the complete machine is powerful in function and higher in practicability; the shell is disposed to be of a bilateral symmetry structure which is in smooth transition, so that multi-angle free massage is effectively realized; and the flexible buffer part is disposed between the support fixing part and the motor fixing seat, so that power stability is realized, meanwhile, noise is effectively reduced, and the sense of use is improved.

### BRIEF DESCRIPTION OF THE DRAWINGS

FIG. 1 is a schematic diagram showing a planar structure of a muscle massage device in a viewing angle state of a front view according to the present application;

FIG. 2 is a schematic diagram showing a planar structure of the muscle massage device in a viewing angle state of a rear view according to the present application;

FIG. 3 is a schematic diagram showing a planar structure of the muscle massage device in a viewing angle state of a left view according to the present application;

FIG. 4 is a schematic diagram showing a three-dimensional structure of the muscle massage device according to the present application;

FIG. 5 is a schematic diagram showing an exploded structure of the muscle massage device according to the present application;

FIG. 6 is a schematic diagram showing a three-dimensional structure of the muscle massage device without a front shell according to the present application; and

FIG. 7 is a schematic diagram showing a three-dimensional structure of a high-frequency hitting unit according to the present application.

### DETAILED DESCRIPTION OF THE INVENTION

Embodiments of the present application will be described in detail, and examples of the embodiments are shown in the accompanying drawings, wherein same or similar numerals throughout indicate same or similar elements or elements with same or similar functions. The embodiments described below with reference to the accompanying drawings are exemplary, are intended to explain the present application, but cannot be understood as restrictions on the present application.

In the description of the present application, it should be understood that a directional or positional relationship indicated by terms such as “center”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise” and “anticlockwise” is based on a directional or positional relationship shown as the accompanying drawings and is merely intended to facilitate describing the present application and simplifying the description, rather than to indicate or imply that the referred device or element has to be located in a specific direction or constructed and operated in the specific direction so as not to be understood as a restriction on the present application.

In addition, terms such as “first” and “second” are used for descriptive purposes only, and cannot be understood as indicating or implying the relative importance or implicitly indicating the number of indicated technical features. Therefore, the features defined as “first” and “second” may explicitly or implicitly include one or more of the features. In the description of the present application, the meaning of “a plurality of” may be two or more unless it may be specifically defined otherwise.

In the present application, terms “mounted”, “connected”, “connection”, “fixed” and the like should be understood in a broad sense unless otherwise specified and defined, for example, “connection” may be fixed connection or detachable connection or integrated connection, may be mechanical connection or electrical connection, may be direct connection or indirect connection through an intermediate medium, and may be internal connection of two elements. For those of ordinary skill in the art, the specific meanings of the above terms in the present application may be understood according to specific situations.

In the present application, that a first feature is located at the “upper” or “lower” a second feature includes that the first feature is in direct contact with the second feature, or the first feature is not in direct contact with the second feature, but is in contact with the second feature by another feature between the first feature and the second feature unless otherwise specified and defined. Moreover, that the first feature is located at the “upper”, “above” and “on” the second feature includes that the first feature is located right above and at the oblique top of the second feature, or only shows that the horizontal height of the first feature is greater than that of the second feature; and that the first feature is located at the “lower”, “below” and “under” the second feature includes that the first feature is located under and at the oblique bottom of the second feature, or only shows that the horizontal height of the first feature is smaller than that of the second feature.

The present application will be further described below in detail by specific implementations in combination with the accompanying drawings.

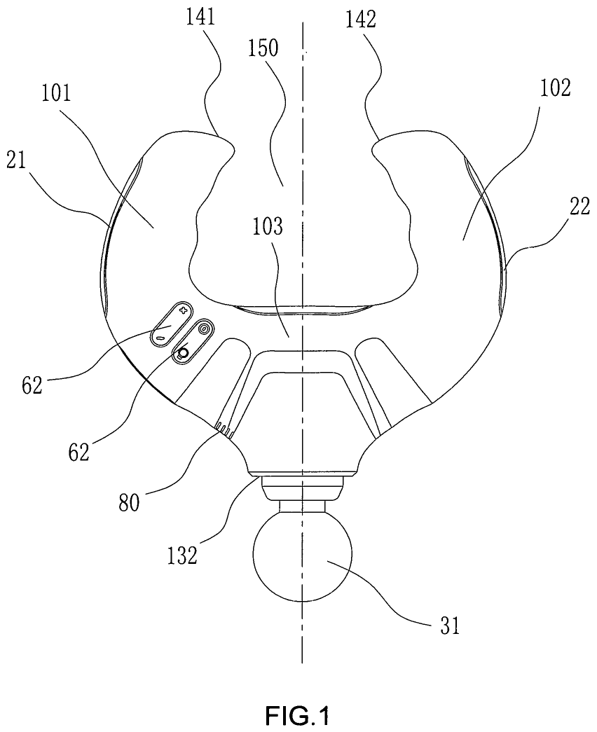

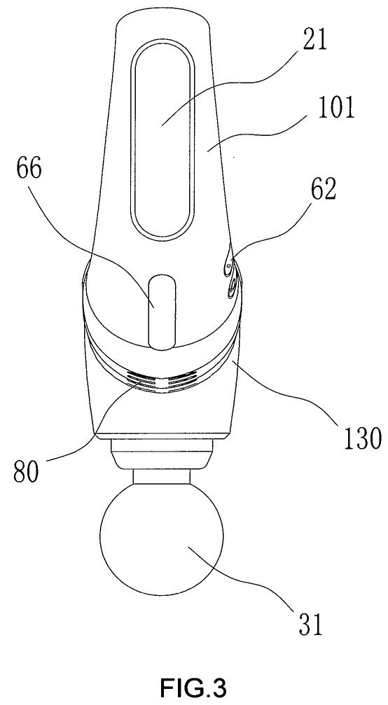

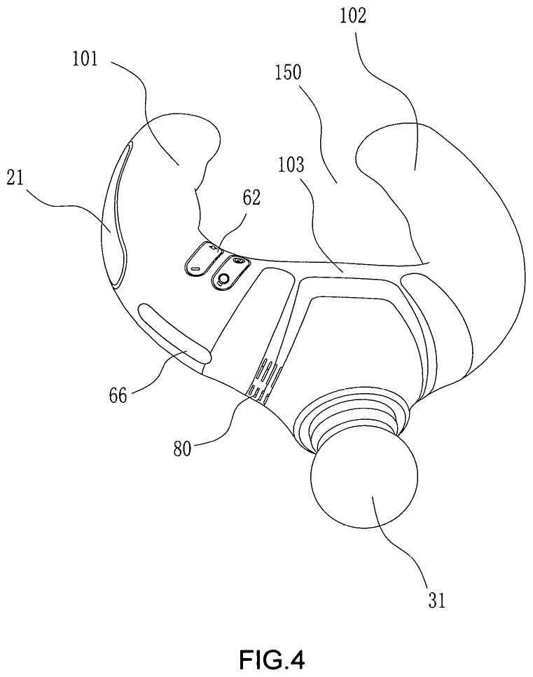

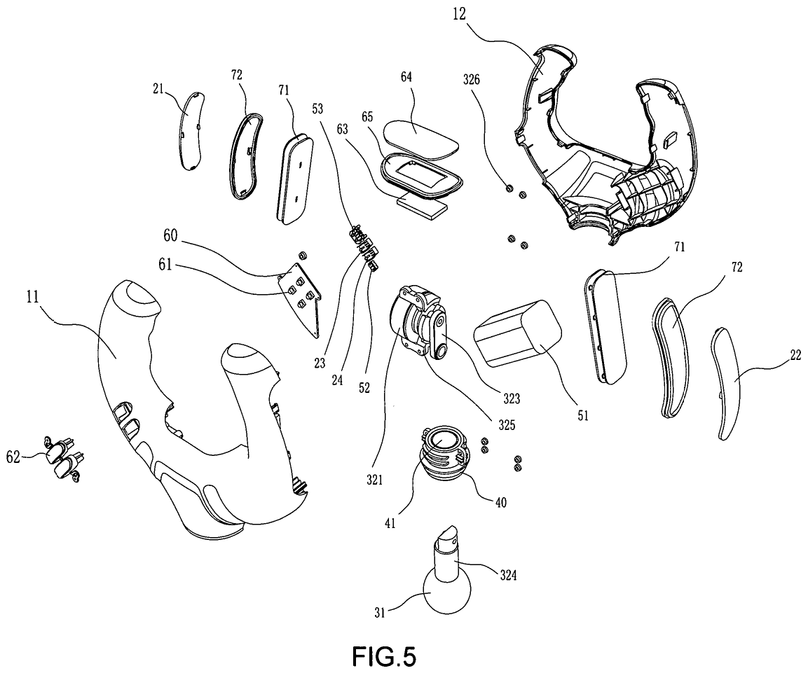

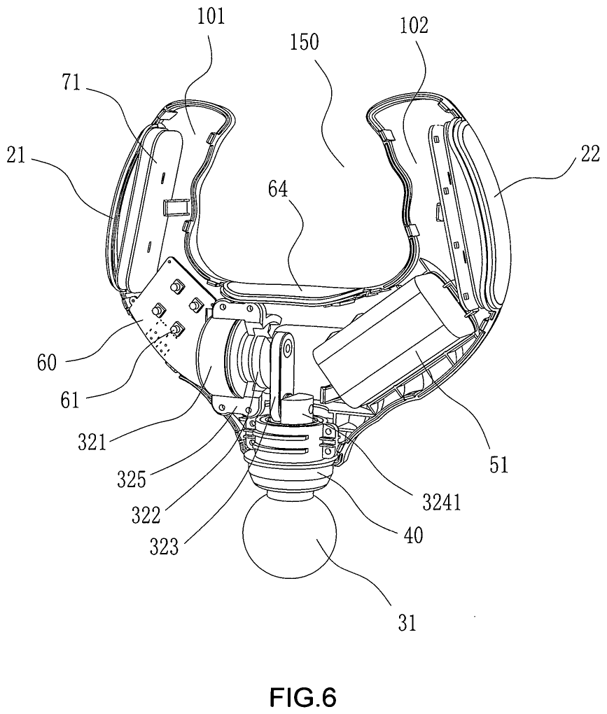

Referring to FIG. 1 to FIG. 7, the present application provides a muscle massage device including a shell and an electric pulse unit, a high-frequency hitting unit, a control unit and a power supply unit which are disposed on the shell. The shell includes a front shell 11 and a rear shell 12 which are matched and abutted, and an internal space of the shell is formed between the front shell 11 and the rear shell 12.

The electric pulse unit stimulates the motion contraction of muscles of a human body by transferring a micro-current signal to the human body according to an EMS electric pulse massage technical principle, thereby achieving the aim of improving the effect on muscle training of the human body. The EMS is short for “Electronic Muscle Stimulation”, namely an electronic muscle stimulation technology.

The electric pulse unit includes a pulse generation mechanism, a first electrode sheet 21 and a second electrode sheet 22. The pulse generation mechanism is disposed inside the shell, and the first electrode sheet 21 and the second electrode sheet 22 are disposed at intervals and are fixedly disposed on the shell. The pulse generation mechanism is configured to generate a pulse current, and the first electrode sheet 21 and the second electrode sheet 22 are connected with the pulse generation mechanism, so that the pulse current may be output to the human body when the first electrode sheet 21 and the second electrode sheet 22 are in contact with the skin of the human body in an electrified state.

In the present embodiment, the shell is provided with a first holding part 101 and a second holding part 102 disposed at intervals, the first electrode sheet 21 is disposed at the first holding part 101, and the second electrode sheet 22 is disposed at the second holding part 102. The first holding part 101 and the second holding part 102 are held by left and right hands of a user, and when the first holding part 101 and the second holding part 102 are respectively held by the left and right hands of the user, the left and right hands of the user are respectively in direct contact with the first electrode sheet 21 and the second electrode sheet 22.

In order to further improve the service performance of the electric pulse unit 20, the electric pulse unit further includes a first electric pulse interface 23 and a second electric pulse interface 24 connected with the pulse generation mechanism, the first electric pulse interface 23 and the second electric pulse interface 24 are configured to be connected with an external electrode sheet so as to further provide a pulse current output for the external electrode sheet.

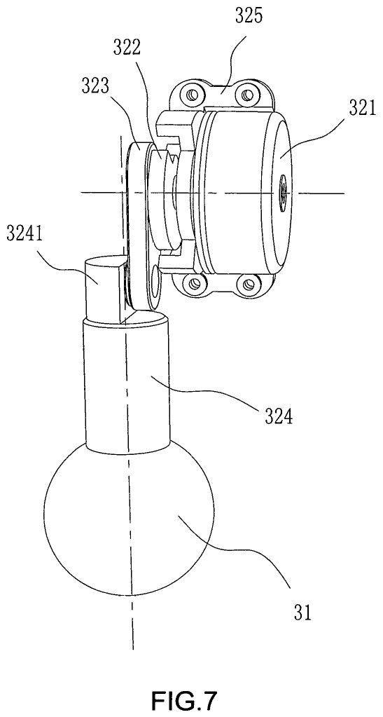

Referring to FIG. 7, the high-frequency hitting unit includes a massage head 31 and a reciprocating driving mechanism. The massage head 31 is at least partially disposed outside the shell so as to be in direct contact with the human body. The reciprocating driving mechanism is disposed inside the shell and is connected with the massage head 31, and the reciprocating driving mechanism is configured to drive the massage head 31 to do high-frequency reciprocating motion, and furthermore, deep hitting massage on muscular fascia of the human body is realized. In the present embodiment, the massage head 31 is removably connected to the reciprocating driving mechanism.

Specifically, the massage head 31 is provided with a massage end for direct contact with the human body and a fixed end for fixed connection with the reciprocating driving mechanism 32. Preferably, there are a plurality of massage heads 31, and structures of a plurality of the massage heads 31 may be set to be the same, partially same or totally different so that the massage heads 31 may be replaced according to use demands of users. During specific implementation, the massage head 31 may be specifically set as a spherical massage head of which the massage end is of a spherical structure, a tapered massage head of which the massage end is of a tapered structure, a flat massage head of which the massage end is of a planar structure, a forked massage head of which the massage end is forked and the like. It may be known that the structure of the massage head 31 is not exhaustively listed in the present application, the massage head 31 may adopt any one existing massage head structure capable of pressing and hitting the muscles of the human body, but is not limited to that in embodiments listed in the present application. Of course, in some embodiments, it is possible to only provide one massage head 31.

The massage head 31 of which the massage end is of the spherical structure is exampled in the accompanying drawing of the embodiment of the present application. If the massage end of the massage head 31 is set to be of the spherical structure, the massage end of the massage head 31 may be wholly made of a flexible material with certain hardness, or the outer surface of the massage end of the massage head 31 is at least coated with a flexible buffer layer with a certain thickness, and furthermore, the comfort level of the massage head 31 in use is improved.

The reciprocating driving mechanism includes a motor 321, an eccentric wheel 322, a linkage rod 323 and a reciprocating part 324.

The motor 321 is fixedly disposed inside the shell and is provided with a motor shaft extending out of a motor main body and capable of rotating. In the present embodiment, the motor 321 preferably adopts a brushless motor which is small in friction force, smooth in operation and low in noise. The eccentric wheel 322 is provided with a pivoting hole, is fixedly connected to the motor shaft by the pivoting hole and is capable of rotating with the motor shaft. The side, far away from the motor main body, of the eccentric wheel 322 is provided with an eccentric shaft, and a central axis of the eccentric shaft is parallel to and deviated from a central axis of the motor shaft. The linkage rod 323 is provided with a first linkage end and a second linkage end, and the first linkage end is rotatably connected to the eccentric shaft. The reciprocating part 324 is provided with a first driving end and a second driving end, and the first driving end of the reciprocating part 324 is fixedly connected to the second linkage end of the linkage rod 323; and the massage head 31 is fixedly connected to the second driving end of the reciprocating part 324. After the motor 321 is electrified, the motor main body drives the motor shaft to rotate, the motor shaft drives the eccentric wheel 322 to rotate, and the linkage rod 323 pivoted to the eccentric shaft of the eccentric wheel 322 does reciprocating motion with the eccentric wheel 322 to further drive the reciprocating part 324 to do reciprocating motion, and finally, the reciprocating motion of the massage head 31 is realized.

The shell is provided with a reciprocating channel on a position corresponding to the reciprocating driving mechanism, and a central axis of the motor shaft is vertical to a central axis of the reciprocating channel. The reciprocating part 324 is at least partially disposed inside the reciprocating channel and is capable of doing reciprocating motion in the reciprocating channel in the axial direction of the reciprocating channel under the drive of the motor 321.

In the present embodiment, the reciprocating part 324 is set to be of a cylindrical structure, the first driving end of the reciprocating part 324 is provided with a fixing lug 3241 in an axial direction of the reciprocating part 324, and the reciprocating part 324 is fixedly connected to the second linkage end of the linkage rod 323 by the fixing lug 3241. The shell is provided with a reciprocating hole in a position corresponding to the reciprocating part 324, the inside of the reciprocating hole is provided with a reciprocating guide part 40 in which a cylindrical through hole 41 is formed, and the cylindrical through hole 41 forms the reciprocating channel.

Further referring to FIG. 7, the reciprocating driving mechanism further includes a motor support 325, and the motor support 325 is configured to fix the motor 321 inside the shell.

The motor support 325 includes a motor accommodating part and a support fixing part.

The motor accommodating part is provided with a concave motor accommodating cavity, and a motor shaft through hole is formed in the middle of the motor accommodating cavity. An inner contour of the motor accommodating cavity is identical with an outer contour of the motor main body, the motor shaft through hole is configured to be penetrated by the motor shaft of the motor 321, the motor shaft is connected with the eccentric wheel 322 after penetrating through the motor shaft through hole, that is, the motor 321 and the eccentric wheel 322 are respectively disposed at two sides of the motor support 325.

The support fixing part is formed by outwards extending from the motor accommodating part. The inside of the shell is provided with a motor fixing seat, and the motor support 325 is fixedly connected to the motor fixing seat by the support fixing part. The shell is provided with heat radiating holes 80 in positions corresponding to the motor 321.

In order to stabilize the motor 321 and prevent the motor 321 from generating high-frequency vibration during work, the support fixing part and the motor fixing seat are matched to be provided with a locking structure, and the locking structure includes at least one first locking notch disposed on the support fixing part and at least one second locking notch disposed on the motor fixing seat, wherein the position of the first locking notch corresponds to the position of the second locking notch, and the first locking notch and the second locking notch support against each other when being fixed.

In order to further improve the stability of the motor 321 and reduce noise generated when the motor 321 works, a flexible buffer part 326 is disposed between the support fixing part and the motor fixing seat. The flexible buffer part 326 has a certain thickness and elasticity, is disposed between the support fixing part and the motor fixing seat to block direct contact between the support fixing part and the motor fixing seat, avoid collision and friction between the support fixing part and the motor fixing seat and effectively reduce the noise, and provides effective vibration absorption for high-frequency work of the motor 321 by virtue of its elastic buffer property.

The high-frequency hitting unit drives the massage head 31 to precisely and deeply vibrate at high frequency by virtue of the reciprocating driving mechanism to impact a target part of a body, stimulate fascia by muscle conduction, activate cells and promote blood circulation, thereby accelerating muscle recovery, reducing lactic acids and relaxing soft tissues of muscles.

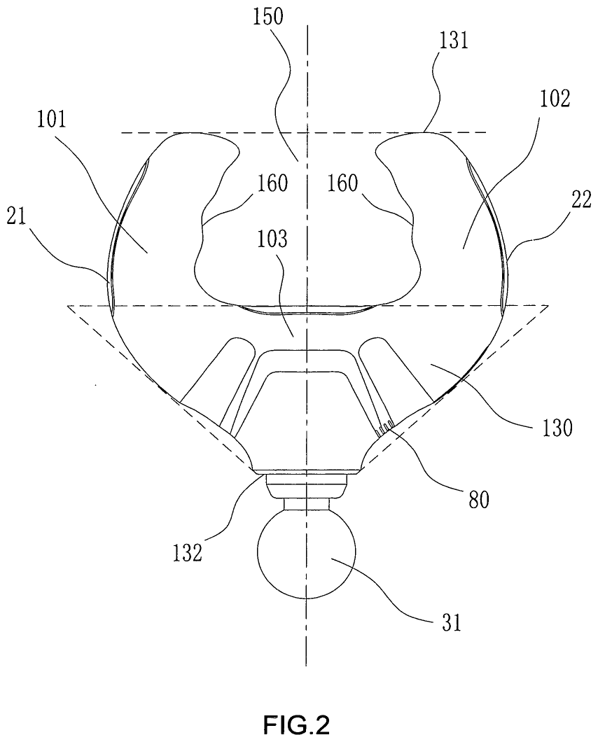

As shown in FIG. 1 to FIG. 2, the shell is provided with a first end 131 and a second end 132 corresponding to each other, the first holding part 101 and the second holding part 102 are located at the first end 131 of the shell, the reciprocating hole is located in the second end 132 of the shell, and the first end 131 of the shell is longer than the second end 132 of the shell.

Referring to FIG. 1 and FIG. 2, the specific structure of the shell of the muscle massage device provided by the present application is further described below in detail by taking a placing angle of the massage head 31 located below the shell as shown in FIG. 1 and FIG. 2 as a reference.

Referring to FIG. 2, in the present embodiment, a shell end shown as a horizontal dotted line at the topmost end in FIG. 2 serves as the first end 131 of the shell, and a main part 130 of the shell is approximately a part circled by the dotted line in FIG. 2. Seen from FIG. 2, the main part 130 of the shell is approximately of an inverted trapezoidal structure which is wide at the upper part and narrow at the lower part. Two outer sides of the upper end of the main part 130 of the shell respectively extend outwards to form a first grip 141 and a second grip 142, and the first grip 141 and the second grip 142 respectively form the first holding part 101 and the second holding part 102. The first electrode sheet 21 and the second electrode sheet 22 are respectively disposed at two outer sides, facing away from each other, of the first grip 141 and the second grip 142, two opposite inner sides of the first grip 141 and the second grip 142 are provided with corrugated structures 160 in smooth transition, and the corrugated structures 160 are suitable for being held by fingers of a human body.

An outer contour of the shell is of an axisymmetric structure in bilateral symmetry relative to a central axis of the reciprocating channel. The first grip 141 and the second grip 142 are symmetrically disposed left and right at intervals. An avoiding gap 150 is formed between the first grip 141 and the second grip 142 at the first end 131 of the shell, and a third holding part 103 corresponding to the massage head 31 is formed at the avoiding gap 150 in the first end 131 of the shell. If the third holding part 103 is used when the high-frequency hitting unit works, a user may hold the muscle massage device with a single hand. The surface of the third holding part 103 is provided with an antiskid structure, or the outer surface of the third holding part 103 is at least made of an antiskid material.

In the present embodiment, the outer contour of the shell is in smooth transition as a whole.

The power supply unit includes a rechargeable battery 51, an electric energy input interface 52 and an electric energy output interface 53.

The rechargeable battery 51 is disposed inside the shell, and the inside of the shell is provided with a battery fixing seat configured to fix the rechargeable battery 51. The electric energy input interface 52 is connected with the rechargeable battery 51 and is configured to charge the rechargeable battery 51. The electric energy output interface 53 is connected with the rechargeable battery 51 and is configured to provide electric energy for external electronic equipment via the rechargeable battery 51.

In the present embodiment, the charging input of the power supply unit may be 5V/2 A, 9V/2 A or 12V/1.5 A; the charging power of the power supply unit is lower than 10 W; and the power supply capacity of the rechargeable battery is 2000 Mah. Of course, in some another embodiments, the power supply unit may also directly use common batteries such as four 16.8V common batteries.

The control unit includes a master control unit, an instruction unit connected with the master control unit and a display unit connected with the master control unit.

The master control unit includes an electric pulse control module configured to control the electric pulse unit to work, a high-frequency hitting control module configured to control the high-frequency hitting unit to work and an electric energy control module configured to control the power supply unit to work.

At least one electric pulse working mode is preset for the electric pulse control module, the electric pulse working mode is embodied as an output mode of the pulse current, meanwhile, a plurality of electric pulse working gears are set for each of the electric pulse working modes, and the electric pulse working gears are embodied as output intensities of the pulse current.

At least one high-frequency hitting working mode is preset for the high-frequency hitting control module, the high-frequency hitting working mode is embodied as a hitting mode of the massage head, and the hitting mode of the massage head is realized in a working manner of the motor 321; and meanwhile, a plurality of high-frequency hitting working gears are further set for each of the high-frequency hitting working modes, and the high-frequency hitting working gears are embodied as the hitting frequency of the massage head, and the hitting frequency of the massage head is controlled by the rotating speed of the motor 321.

In the present embodiment, standby power consumption of the muscle massage device is lower than 50 uA, and the working temperature of the muscle massage device is 10 DEG C. below zero to 40 DEG C. The pulse frequency of the electric pulse unit is 7K, and the pulse voltage of the electric pulse unit is lower than 200V. The electric pulse control module is provided with 6 different electric pulse working modes and 15 electric pulse working gears with different-intensity outputs with respect to the electric pulse unit; and the high-frequency hitting control module is provided with one high-frequency hitting working mode with respect to the high-frequency hitting unit. Of course, in some another embodiments, another number of working modes and working gears may be set for the electric pulse control module and the high-frequency hitting control module according to actual performance demands.

The instruction unit includes a mode switching module and a gear adjusting module. The mode switching module is configured to control the electric pulse control module or the high-frequency hitting control module to be started and control the electric pulse control module or the high-frequency hitting control module to work according to a preset working mode. The gear adjusting module is configured to adjust a working gear of the electric pulse control module or the high-frequency hitting control module in the current working mode, and the gear adjusting module includes a working intensity increasing instruction and a working intensity reducing instruction.

The display unit includes a working mode display module, a work parameter display module and an electric energy display module.

The working mode display module is configured to embody a currently selected working mode. The working mode display module includes a code display module and a light emitting display module. The code display module may adopt a digital code and/or letter code. In the present embodiment, an English code “EMS” for representing the electric pulse control module and an English code “MASSAGE” for representing the high-frequency hitting control module are preset; if the electric pulse control module is started, the English code “EMS” is lightened; and if the high-frequency hitting control module is started, the English code “MASSAGE” is lightened. Meanwhile, the muscle massage device is respectively provided with a group of light emitting units on positions corresponding to the first electrode sheet 21 and the second electrode sheet 22, the light emitting units include light emitting circuit boards 71 and light guide plates 72, and the light emitting units are connected with the light emitting display module so as to work under the control of the light emitting display module. In the present embodiment, the light emitting units are disposed on the first electrode sheet 21 and the second electrode sheet 22, the light guide plates form light guide regions at the peripheries of the first electrode sheet 21 and the second electrode sheet 22, LED lamps on the light emitting circuit boards 71 may emit two rays with different colors, such as a blue ray and a white ray; if the electric pulse control module is started, the light emitting units project the blue ray at the peripheries of the first electrode sheet 21 and the second electrode sheet 22; and if the high-frequency hitting control module is started, the light emitting units project the white ray at the peripheries of the first electrode sheet 21 and the second electrode sheet 22. It may be understood that, in some another embodiments, the light emitting colors of the LED lamps may be set as any two existing colors, but are not limited to those in the embodiments listed in the preset application; and the positions of the light emitting units may also be adjusted according to actual design demands, for example, the light emitting units are disposed at the ends of the first grip 141 and the second grip 142.

The working parameter display module is configured to embody working parameters related in the current working mode, such as a working gear and other information. The electric energy display module is configured to embody working information of the power supply unit, such as a working state, current electric quantity and other information of the rechargeable battery 51.

In the present embodiment, the inside of the shell is provided with a master control circuit board 60, the control unit, the pulse generation mechanism, the first electric pulse interface 23, the second electric pulse interface 24, the electric energy input interface 52 and the electric energy output interface 53 are all disposed on the master control circuit board 60, and the light emitting circuit boards 71 are connected with the master control circuit board 60. The master control circuit board 60 is provided with buttons 61 configured to send control instructions to the instruction unit, and the shell is provided with button holes in positions corresponding to the buttons 61 and is provided with button shells 62. In the present embodiment, there are four buttons 61 in total, which are respectively a power on/off button, a mode switching button, a gear increasing button and a gear decreasing button. The inside of the shell is further provided with a display screen 63 connected with the display unit, the shell is provided with a display hole in a position corresponding to the display screen 63 and is provided with a display panel 64, and the display screen 63 and the display panel 64 are fixedly connected to the shell by a display screen fixing seat 65. In the present embodiment, the display screen 63 is disposed in the middle of the shell at the avoiding gap 150. The first electric pulse interface 23, the second electric pulse interface 24, the electric energy input interface 52 and the electric energy output interface 53 are disposed in the same region, the shell is provided with a splicing hole in a position corresponding to the above-mentioned interface device, a splicing end of the interface device is exposed outside the shell, the shell is further provided with an interface cover 66 on a position corresponding to the splicing hole, and the interface cover 66 is capable of opening or closing the splicing hole.

A using method of the muscle massage device provided by the present application will be further described below in detail with reference to one of the embodiments of the muscle massage device provided by the present application.

The muscle massage device is used in a non-charging mode, and the high-frequency hitting unit and the electric pulse unit are not allowed to work at the same time.

If the power on/off button is pressed once, the control unit and the power supply unit are powered on, and the high-frequency hitting control module is defaulted to be started during power-on, which shows that the English code “MASSAGE” of the high-frequency hitting control module is lightened; and the light emitting units project the white ray at the peripheries of the first electrode sheet 21 and the second electrode sheet 22. Of course, in another embodiment, a starting item defaulted during power-on may also be set as the electric pulse control module.

If the mode switching button is pressed for a long time, the mode switching module controls the electric pulse control module to be started, which shows that the English code “EMS” of the electric pulse control module is lightened, and the light emitting units project the blue ray at the peripheries of the first electrode sheet 21 and the second electrode sheet 22. In a similar way, if it is necessary to switch the electric pulse control module to the high-frequency hitting control module, the mode switching button is only required to be pressed for a long time.

In the case of the high-frequency hitting control module, display of the currently selected working mode and working parameters of the high-frequency hitting control module on the display screen 63 may be set in a way that the massage intensity in the current working mode is displayed at the right of the display screen 63; the working gear in the current working mode of the high-frequency hitting control module may be adjusted by the gear adjusting module; the control unit is automatically powered off after the intensity, displayed on the display screen 63, of the high-frequency hitting control module is 0 and is lasted for 30 s; and the control unit is automatically powered off after the high-frequency hitting control module continuously works for 15 min.

In the case of the electric pulse control module, display of the currently selected working mode and working parameters of the electric pulse control module on the display screen 63 may be set in a way that the working mode of the current electric pulse control module is displayed at the left of the display screen 63, and the working gear, that is, the massage intensity, in the current working mode is displayed at the right of the display screen 63; the working mode and intensity of the electric pulse control module may be switched by transitorily pressing the mode switching button, the gear increasing button or the gear decreasing button as required, the corresponding display module on the display screen 63 flickers in a selected state, after setting is completed, automatic affirmation is performed within 5 s, and the corresponding display module on the display screen 63 stops flickering; the control unit is automatically powered off after the intensity, displayed on the display screen 63, of the electric pulse control module, is 0 and is lasted for 30 s; if the left and right hands of the human body are in contact with the first electrode sheet 21 and the second electrode sheet 22 in the working state of the electric pulse control module, the English code “EMS” is in a long-time lighting state; and if the left and right hands of the human body leave from the first electrode sheet 21 and the second electrode sheet 22 for 30 s, the control unit is automatically powered off.

If the control unit is not operated by any person in a power-on state for over 30 min, the control unit is automatically powered off.

Electric quantity prompt and low power caution: the display screen 63 is provided with a charging indication lamp configured to indicate that the rechargeable battery 51 is in a charging state and an electric quantity indication lamp configured to indicate the electric quantity of the rechargeable battery 51. When a data line is inserted to the electric energy input interface 52 to charge the rechargeable battery 51, the charging indication lamp and the electric quantity indication lamp are only lightened, but other buttons and functions are turned off; when the rechargeable battery 51 supplies power, the electric quantity indication lamp displays the current electric quantity; when the electric quantity of the rechargeable battery 51 is lower than 10% in a working state, the electric quantity indication lamp rapidly flickers; when the electric quantity of the rechargeable battery 51 is lower than 1%, automatic power-off is performed; and after charging starts, power-on is allowed only when the electric quantity of the rechargeable battery 51 is higher than 5%.

In the description of the present specification, the description of reference terms such as “one implementation”, “some implementations”, “one embodiment”, “some embodiments”, “example”, “specific example” or “some examples” means that specific features, structures, materials or characteristics described in combination with the embodiment or example are contained in at least one embodiment or example of the present application. In the present specification, the schematic representation of the above terms does not necessarily refer to the same embodiment or example. Moreover, the described specific features, structures, materials or characteristics can be combined in an appropriate manner in any one or more embodiments or examples.

The above content is the further description of the present application in combination with specific implementations, but cannot affirm that the specific implementation of the present application is only limited on these descriptions. Several simple derivations or replacements may be further made by those of ordinary skilled in the art in the technical field of the present application without departing from the concept of the present application.

What is claimed is:

1. A muscle massage device, comprising:
a shell;
a high-frequency hitting unit, disposed on the shell;
an electric pulse unit, disposed on the shell; and
a control unit, disposed on the shell and connected with the high-frequency hitting unit and the electric pulse unit, wherein the control unit optionally controls the high-frequency hitting unit and the electric pulse unit to work according to a preset mode, and the high-frequency hitting unit and the electric pulse unit are not allowed to work at the same time;
the shell is provided with a first holding part and a second holding part disposed at intervals; the electric pulse unit comprises:
a pulse generation mechanism, disposed inside the shell;
a first electrode sheet, disposed on the first holding part; and
a second electrode sheet, disposed on the second holding part, wherein the pulse generation mechanism is connected with the first electrode sheet and the second electrode sheet, and a pulse current is generated and output when the first electrode sheet and the second electrode sheet are in contact with the skin of a human body after being electrified;
the high-frequency hitting unit comprises:
a massage head, at least partially disposed outside the shell; and
a reciprocating driving mechanism, disposed inside the shell and connected with the massage head;
the shell is provided with a reciprocating channel on a position corresponding to the reciprocating driving mechanism; and the reciprocating driving mechanism is configured to drive the massage head to do high-frequency reciprocating motion in an axial direction of the reciprocating channel.

2. The muscle massage device according to claim 1, wherein the first electrode sheet and the second electrode sheet are respectively disposed at outer sides of the first holding part and the second holding part; and inner sides of the first holding part and the second holding part are provided with corrugated structures suitable for being held by fingers of a human body.

3. The muscle massage device according to claim 2, wherein the shell is provided with a first end and a second end corresponding to each other, the first holding part and the second holding part are located at the first end of the shell and are disposed left and right at intervals, the reciprocating channel is located at the second end of the shell, and an outer contour of the shell is of an axisymmetric structure in bilateral symmetry relative to a central axis of the reciprocating channel.

4. The muscle massage device according to claim 3, wherein the first end of the shell is provided with a first grip and a second grip, the first grip and the second grip respectively form the first holding part and the second holding part; and an avoiding gap is formed between the first grip and the second grip at the first end of the shell, and a third holding part corresponding to the massage head is formed at the avoiding gap in the first end of the shell.

5. The muscle massage device according to claim 4, wherein the reciprocating driving mechanism comprises:
a motor, fixedly disposed inside the shell, wherein the motor comprises a motor main body and a motor shaft extending out of the motor main body and capable of rotating, and a central axis of the motor shaft is vertical to the central axis of the reciprocating channel;
an eccentric wheel, fixedly connected to the motor shaft, wherein the side, far away from the motor main body, of the eccentric wheel is provided with an eccentric shaft, and a central axis of the eccentric shaft is parallel to and deviated from the central axis of the motor shaft;
a linkage rod, wherein the linkage rod is provided with a first linkage end and a second linkage end, and the first linkage end is rotatably connected to the eccentric shaft; and
a reciprocating part, wherein the reciprocating part is provided with a first driving end and a second driving end, and the first driving end of the reciprocating part is fixedly connected to the second linkage end of the linkage rod; the massage head is fixedly connected to the second driving end of the reciprocating part; and the reciprocating part is at least partially disposed in the reciprocating channel and is capable of doing reciprocating motion in the axial direction of the reciprocating channel under the drive of the motor.

6. The muscle massage device according to claim 5, wherein the reciprocating driving mechanism further comprises a motor support configured to fix the motor inside the shell; the motor support comprises:
a motor accommodating part, wherein the motor accommodating part is provided with a motor accommodating cavity and a motor shaft through hole, an inner contour of the motor accommodating cavity is identical with an outer contour of the motor main body, and the motor shaft through hole is configured to be penetrated by a motor shaft; and
a support fixing part;
the inside of the shell is provided with a motor fixing seat, the motor support is fixedly disposed on the motor fixing seat by the support fixing part, and a flexible buffer part is disposed between the support fixing part and the motor fixing seat.

7. The muscle massage device according to claim 6, wherein the support fixing part and the motor fixing seat are matched to be provided with a locking structure, and the locking structure comprises:
at least one first locking notch disposed on the support fixing part; and
at least one second locking notch disposed on the motor fixing seat, wherein the position of the first locking notch corresponds to the position of the second locking notch, and the first locking notch and the second locking notch support against each other.

8. The muscle massage device according to claim 4, wherein the electric pulse unit further comprises a first electric pulse interface and a second electric pulse interface connected with the pulse generation mechanism, the first electric pulse interface and the second electric pulse interface are configured to be connected with an external electrode sheet so as to further provide a pulse current output for the external electrode sheet.

9. The muscle massage device according to claim 4, further comprising a power supply unit connected with the control unit, wherein the power supply unit comprises:
a rechargeable battery disposed inside the shell;
an electric energy input interface connected with the rechargeable battery; and
an electric energy output interface connected with the rechargeable battery.

10. The muscle massage device according to claim 4, wherein the control unit comprises:
a master control unit;
an instruction unit connected with the master control unit; and
a display unit connected with the master control unit;
the master control unit comprises:
an electric pulse control module configured to control the electric pulse unit to work, wherein at least one electric pulse working mode is preset for the electric pulse control module, the electric pulse working mode is embodied as an output mode of the pulse current, a plurality of electric pulse working gears are set for each of the electric pulse working modes, and the electric pulse working gears are embodied as the output intensity of the pulse current; and
a high-frequency hitting control module configured to control the high-frequency hitting unit to work, wherein at least one high-frequency hitting working mode is preset for the high-frequency hitting control module, the high-frequency hitting working mode is embodied as a hitting mode of the massage head, a plurality of high-frequency hitting working gears are further set for each of the high-frequency hitting working modes, and the high-frequency hitting working gears are embodied as the hitting frequency of the massage head;
the instruction unit comprises:
a mode switching module, configured to control the electric pulse control module or the high-frequency hitting control module to be started and control the electric pulse control module or the high-frequency hitting control module to work according to a preset working mode; and
a gear adjusting module, configured to adjust a working gear of the electric pulse control module or the high-frequency hitting control module in the current working mode;
the display unit comprises:
a working mode display module, configured to embody a currently selected working mode; and
a working parameter display module, configured to embody working parameters in the current mode.
